(19) United States
(12) Patent Application Publication
(10) Pub. No.: US 2002/0127732 A1
(43) Pub. Date: Sep. 12, 2002

(54) Process for the diagnosis of diseases, pathological states and pathophysiological as well as physiological measurement units

(76) Inventors: Gotz Nowak, Erfurt (DE); Elke Bucha, Erfurt (DE)

Correspondence Address: KAGAN BINDER, PLLC, Suite 200, Maple Island Building, 221 Main Street North, Stillwater, MN, 55082, US

(21) Appl. No.: 10/014,389

(22) Filed: Oct. 22, 2001

(30) Foreign Application Priority Data
Apr. 25, 2000 (EP) PCT/EP00/03690
Apr. 23, 1999 (DE) 199 18 569.7

(51) Int. Cl. G01N 33/00; G01N 33/53

(52) U.S. Cl. 436/128; 436/129; 436/165; 436/501; 422/55; 422/58; 422/102; 435/4; 435/7.1

(57) ABSTRACT

The invention relates to a process for the diagnosis of illnesses, pathologic states as well as physiological measurement units, which is based on the immobilization of recognition structures by means of a specific linker at specific functional polymer surfaces.

Processes for the easy and fast measurement of pathological states, and also of pathophysiological and physiological data of the organism, using the interaction of functional plastic surfaces having specific linkers, as described in WO 98/46648, are the subject-matter of the invention.

The management used at the moment for the quantification of parameters to be diagnosed which are present in the organism partly in ng-amounts or picomolar concentrations only, becomes more and more expensive and the methods used therein are costly and time-consuming. For example, the ELISAs, which have long since been in use, and methods derived therefrom are characterized in that they actually detect relatively small amounts of substance, but demand unreasonably high expenditure and involve a lot of time.

The reason for this is the fact that the substance to be detected has to be recognized specifically by means of specific antibodies which were produced in organisms external to the human body against protein-like substances of the human being. The detection of this specific external protein antibody is effected for example by means of an unspecific further antibody directed against the protein of the animal, to which a corresponding recognition structure was bonded, which is quantified by means of most modern detection methods, essentially radioactive methods, and also spectrophotometrically detectable measurement methods. For the detection, costly diagnosis equipment is used, such as ELISA-readers, spectrophotometers, liquid-scintillation-counters and others, which additionally demand relatively high expenditure and a lot of staff to run and service them.

Thus, the objective of the present invention was to provide semi-quantitative, and also quantitative detection methods by means of a specific form of interaction, which deliver results within a few minutes, whose exactness is comparable to the methods described above, so that a new quality of bed-side-diagnostics can be expected. To achieve this, the inventive methods use the principle described in WO 98/46648 of immobilizing specific recognition structures, such as antigens, specific antibodies, and also enzymes or inhibitors of enzymes, by means of a linker on polymer surfaces.

Upon consideration of this principle, new methods of diagnosing illnesses, body function disorders, and also physiological or pathophysiological measurement data could be developed. Such measurements concerning the functioning or disorder of the body function are used in the laboratory chemical, microbiological or enzymological diagnostics in human medicine, and also in veterinary medicine as well as in the biological sciences.

The described immobilization of recognition structures allows the fast, semi-quantitative determination of substances by means of a competition mechanism described in detail in the following. Surprisingly, it has also been found that immobilization according to this method entails an extraordinarily high occupation by the substance to be diagnosed. As a consequence, the use of common absorption methods, such as UV/Vis or IR-absorption spectroscopy is made considerably easier.

As described in WO 98/46648, molecular recognition structures, which can have highly specific interaction with substances to be diagnosed, are bonded to a linker comprising a structural element having the ability to form hydrogen bridges for the objective of the present invention.

Linkers which can be used for the disclosed methods are molecules having at least two functional groups L1 and L2. One of these functional groups (L1) has to be able to form hydrogen bridges and thus allow the linker to be bonded to the polymer surface. The functional group L2 is chosen in such a way that a bond between the linker and the substance to be immobilized can be created. Several linkers having different groups L2 can be simultaneously used to simultaneously apply several substances to the polymer surface. However, it is also possible to use linkers of a type having several groups L2 which are the same or different. Linkers having several equivalent or different groups L1 can equally be used. Preferably, L1 and L2 are linked by an alkyl chain or a polyether.

Structural element L1 is preferably a polar hydrogen atom, as, for example, present in OH, SH, NH or PH bonds. This structural element is preferably present at a sufficiently water soluble compound as a linker further carrying the structural element L2. L1 applied terminally to the linker is particularly preferred.

The functional group by means of which the substance can, preferably covalently, be bonded to the linker (L2) is for example a succinimidyl succinate, succinimidyl propionate, nitrophenyl carbonate, trisylate, epoxide, aldehyde, isocyanate or a maleinimide.

Functional groups L2 by means of which the preferred linkers can be modified to bond a substance are, e.g., described in the catalogue of the company Shearwater Polymers, Inc., 2307 Spring Branch Rd., Huntsville, Ala. 35801 (USA).

Polyalkylene glycols, polyalkylene imines, polyalkylene amines or polyalkylene sulfides as well as polyoxazillines can preferably be used as linkers, polyalkylene glycols being particularly preferred. The use of polyethylene glycols (PEG) is particularly preferred. The mentioned compounds preferably have a molecular weight of 0.5-50 kDa.

Functional polymer surfaces that can be used for the inventive methods are also described in WO 98/46648. Homo- or copolymers are used, for the preparation of which at least one monomer type is used which contains, besides a polymerizable double bond or a polycondensable functional group, a further carbonyl group in the form of a ketone or a carbonic acid derivative, which does not participate in the polymerization reaction. Preferably, the polymer contains a structural element of formula (A);

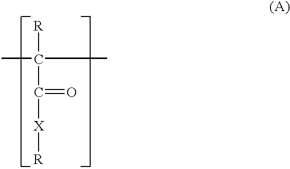

wherein R can be the same or different and can be an alkyl or aryl group or a hydrogen atom. The alkyl group can be linear or branched and preferably consists of 1 to 20 carbon atoms. The aryl group preferably consists of 6 to 18, more preferably of 6 to 12 carbon atoms. X is optional and is O, N or CH₂. In case X=N, N has, in addition to the one indicated in formula (A), a further group R, which is independently of the other groups R defined as above.

Particularly preferred alkyl groups are a straight-chained or branched, optionally substituted C₁₋₈ alkyl group, such as a methyl, ethyl or propyl group. Examples of optionally present substituents comprise one or more halogen atoms, such as fluorine, chlorine, bromine or iodine atoms or hydroxyl groups, C₁₋₆ alkyl or C₁₋₆ alkoxy or C₁₋₆ alkylthiol groups. A particularly preferred aryl group is a monocyclic or bicyclic, optionally substituted aryl group which can optionally comprise one or more heteroatoms. Phenyl, 1- or 2-naphthyl, indenyl or isoindenyl groups are examples of such aryl groups. C₃₋₉ heteroaryl groups, containing heteroatoms selected from the group consisting of oxygen, sulfur or nitrogen atoms, are an example of aryl groups containing heteroatoms. Monocyclic heteroaryl groups comprise for example pyrolyl, furyl, thienyl, imidazolyl, N-methylimidazolyl, N-ethylimidazolyl, benzothiazolyl, quinazolinyl, naphthylpyridinyl, quinolinyl, isochinolinyl and tetrazolyl.

A preferred polymer containing such groups is a polyalkyl methacrylate (PAMA) having an alkyl group preferably comprising 1-6 C-atoms, such as polymethylmethacrylate (PMMA), polyethylmethacrylate (PEMA) or polypropylmethacrylate. Furthermore, polyvinyl acetate, polycyclohexylmethacrylate or polyphenylmethacrylate can be used. The use of polymethylmethacrylate is particularly preferred.

Copolymers or polymer mixtures of facultative portions of the aforementioned polymers among themselves or with one or more further polymer components, such as polystyrene, polyacrylonitrile or polyamides can be used as well. Preferably, the portion of the monomers comprising a structural element (A) in such mixed polymers is at least 20%, more preferably at least 40% and most preferably at least 60%.

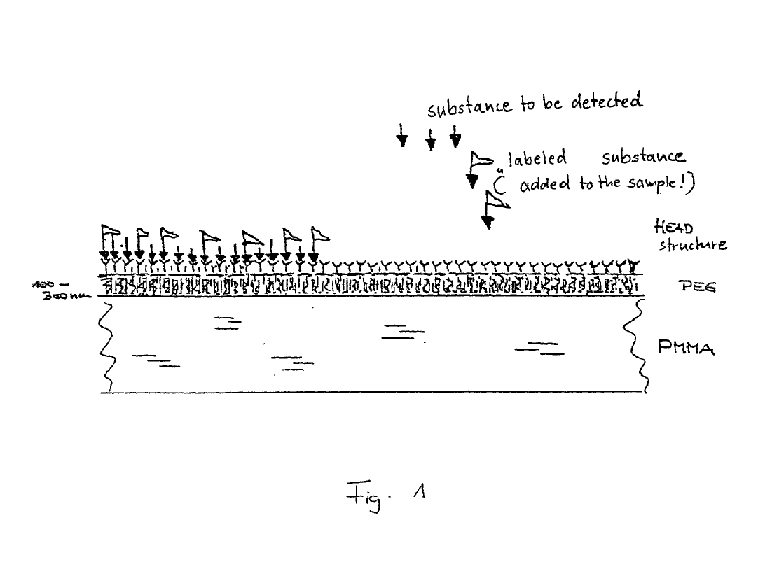

To carry out the inventive diagnosis processes, microtiter plates, cuvettes or measuring tubes for diagnostical analysis devices consisting of the described polymers are used. Structures forming stable bonds with the linkers are present on the surface of those test receptacles. Surprisingly, the bond is effected upon mere contact of the linkers with the polymer surface, without the necessity of increased temperatures or the use of catalysts or other reaction accelerating reagents. The resulting bond is of excellent stability and cannot be broken in aqueous solutions by varying the pH in a range of 2 to 13. The bond is also resistant to rinsing with salt solutions of high ionic strength (2n glycine, 2n urea). Thus, linker-coupled specific recognition structures such as antigens, antibodies, enzymes or other special counter actors to a substance that is to be diagnosed can be applied to the polymer surface. The unit area bonding density at the surface is remarkably high. When PAMA and PEG are used, layers having a thickness of 100-300 nm are formed depending on the respective PEG chain length (see FIG. 1). These layers do not affect the transparency of the polyalkyl methacrylate material used, and, consequently, the transmission of visible, ultraviolet or infrared light is, as a rule, not disturbed. The specific recognition structures bonded terminally to the linkers do not have a significant influence on the light transmission, either. Due to the high surface density of the bonded bio molecules the direct exact measurement of the substances to be detected in blood, plasma or other body fluids is possible. The measurement can therefore be effected by means of absorption measurements at a wave length which is characteristic of the substance to be diagnosed and which should be within the UV, visible or IR-range of the spectrum.

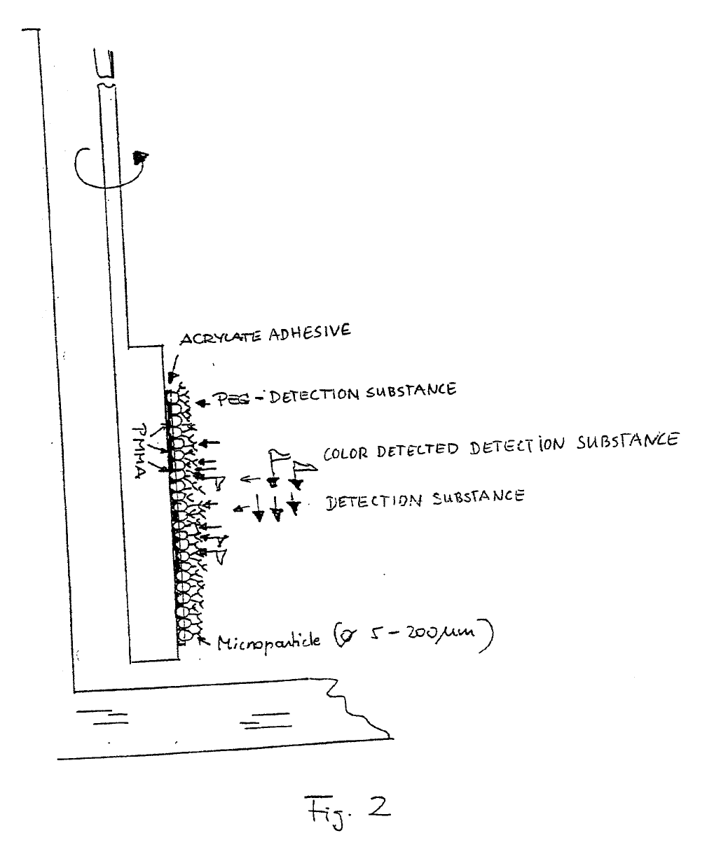

The new diagnosis method used allows a further original detection process. After the recovery of the test fluid to be used or during preparation of the sample, a defined quantity of the substance to be detected is added to the measurement sample. Specific marking and detection agents are bonded to this added detection substance. As a rule, they are dyes, preferably intensive vital-dyes or radioactive markers which are either qualitatively detected by the unaided eye or in connection with certain wave lengths in photometers, spectrophotometers, fluorescence measurement devices, luminescence measurement devices, and also in liquid scintillation counters or γ-counters. The added defined amount of the labeled interaction partner should occupy a certain part of the bonding sites present on the polymer surface which is covered with linkers and recognition structures. This ensures that the unknown quantities of the substance to be detected present in the blood compete with the specifically dyed detection substance for the occupation of the bonding sites. This competition mechanism is exactly quantifiable and linearly correlated in ranges of at least between 25 and 200% of the normal value, so that the sought concentration of the added detection substance can thus be between 25 to 200%. The more of the bonding partner which is to be detected is present in the examined body fluid, the less of the added labeled interaction partner is bonded. Through this process, it is possible to carry out quantitative examining processes by means of the above-mentioned detection methods, and also to provide ultra fast, semi-quantitative tests in the form of a color comparator test. For this bed-side application, it is no longer a microtiter plate or a reaction receptacle which are coated on the same basis as described above, but stirring devices, such as paddle, whirl, spatula or similar things, which consist of functional polymer, are used (FIG. 2). Furthermore, monodisperse microparticles consisting of these kinds of plastic, such as polyalkyl methacrylate (Ø5-200 μm), can be used, which are in a single layer adhered to any plastic surface by means of suitable adhesives, preferably monoacrylate adhesive. This process is also suitable for the use of reaction receptacles, microtiter plates or stirring devices which do not consist of the above-described functional polymers, but which are only coated with such polymers in an additional process step.

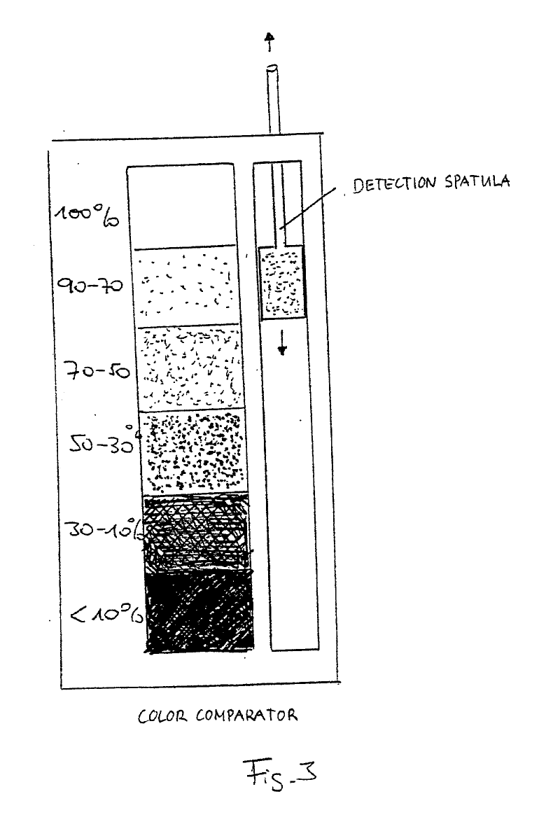

The stirring devices, which should preferably have planar surfaces in order to allow an easy analysis, are equipped with linker-coupled recognition structures during production or before they are used. When doing so, a defined amount of these bonding partners has to be applied to the surface. A defined amount of bonding partner, which is labeled by color in a visible range, (preferably red, blue or black) is added to the sample. After this pre-analytic process, the coated spatula or the like is stirred, pivoted or flexibly contaminated in the body fluid (blood, plasma, urine or the like) for a defined period of time, and subsequently freed from possible blood components or disturbing color changes by means of a washing or rinsing step. After that, the dyed measurement surface of the stirring device is compared as to the intensity of the detection color by means of a color comparator test (color comparison test) (FIG. 3). Before that, the gradual color scale has been quantified by means of color intensity gauging of the system. Through this, well graded quantity steps of the substance to be detected are obtained in the color comparator test and a largely quantitative statement about the amount of substance in the solution can be made.

For a bed-side diagnosis it should be ensured that the areas essential for this use (subtherapeutic, therapeutic, toxic or critically toxic) can be analyzed by three or four color steps at the most. Alternatively, other quantity steps adapted to the clinical circumstances can be included in the test. This semi-quantitative method is designed as bed-side method in such a way that it can be carried out everywhere as single or one-step method without the use of auxiliaries within a few minutes, only by carrying out an easy dilution step of the corresponding starting solution, which can be realized by any unskilled person (“Point-of-care method”).

Such single-step-diagnosis processes as well as the provision of the used equipment are further explained by way of example with reference to a polyethylene glycol/polyalkyl methacrylate system.

1) Methods for the polyethylene glycol coupling of anti-bodies, antigens, active substances, nucleic acids or parts of DNA, rDNA, mDNA and other active substances intended for diagnostics are for example described in J. Milton Harris (Ed.): Polyethylene glycol chemistry, 1992 Plenum Press, New York; or in Zalipsky S: Chemistry of polyethylene glycol conjugates with biologically active molecules, Advanced Drug Delivery Reviews 16: 157-182, 1995.

2) Production or coating of the receptacles used in diagnostics (cuvettes, reaction receptacles), and also stirring devices, spatulas, slides and other things

Polymethylmethacrylate is the preferred functional polymer material, since all receptacles consisting of this material are commercially available and can in most cases be used immediately and without any further treatment. For special applications, however, the surface can additionally be modified by means of known plasma etching processes in order to allow a better bond of polyethylene glycol-bonded substances on this PMMA surface. But normally it is sufficient to use the smooth unchanged surface of the PMMA materials. If other polymers, or glass, are used, the method of coating the surface by means of immobilizing microparticles is chosen. For this, porous, monodisperse polyalkyl methacrylate particles are used, but preferably polymethylmethacrylate or polybutylmethacrylate particles should be used. A common monoacrylate adhesive, which is treated with high-volatile solvents to obtain the defined flowability, is applied to the surface to be coated and then is coated with one single layer of microparticles by means of the fluidized-bed or powder-shifting method (see FIG. 2). This method has the advantage that, due to the defined surface application of the adhesive, an exact number of polyalkyl methacrylate microparticles is adhered thereto and, thus, also an exactly limited surface allocation for the bonding process can be achieved. Known amounts of recognition structures can be adhered to the specific bonding sites of the polyalkyl methacrylate on the microparticle layer which is then completely saturated by means of suitable additives to the polyethylene glycol-coupled bonding partners (e.g. pure polyethylene glycol).

3) Analysis:

0.2 ml of 3% Na-citrate are filled in a blood-taking syringe or monovette. In addition, 2 ml of a phosphate buffer are added, containing 50 nmol of the protein to be detected, which has been dyed with acridine red or the like. After taking 1 ml of full blood and mixing it with the mentioned sample, the pre-analysis is completed. Subsequently, the amount of the sample mixture required for the measuring process is either used for quantitative of bed-side processes. In quantitative measurement processes, a suitable amount of the sample mixture is filled into the respective cavities of the microtiter plate, in a reaction tube for a photometer or into a cuvette for a spectrophotometer, which has previously been specifically treated according to one of the above-described processes. It is important that the applied amount of anti-bodies, e.g. against fibrinogen, is specifically defined on the PMMA-measurement equipment in order to draw up a quantifiable interaction protocol. After a defined time of shaking and mixing, the measuring cuvette or the microtiter plate is rinsed with buffer, as a rule three times if microtiter plates are used, and if cuvettes and measuring tubes are used, they should be rinsed at least twice with the tenfold volume based on the used detection amount.

Subsequently, a correspondingly compatible detection method (microtiter plate reader, analysis automatic machine, photometer) can be used. For the quantification of the experimental set-up, the usual dose-effect-measurement possibilities are available (calibration curves, digital measurement value registration and data processing).

The semi-quantitative direct measurement in the one-step method suitable for bed-side application is carried out as follows: The sample mixture is filled into a suitable reaction receptacle. After that, the stirring device coated with PAMA (spatula, stirring pole, slide or the like), which the specific reaction partner is strongly bonded to, is moved in this sample mixture for 5 (10) minutes, then filled into a buffer reservoir, which is intended for rinsing or washing, washed there and, finally, quantified by means of a color comparator.

1. A diagnosis process comprising bringing into contact a liquid sample to be examined with the surface of a polymer on which recognition structures are immobilized which are able to bond specific components of the liquid, characterized in that the polymer surface comprises carbonyl groups in the form of keto groups or carboxylic acid derivatives, the recognition structures are immobilized by means of a linker, and that a labeled substance, which can also be bonded by means of the recognition structure, is added to the sample, wherein the addition is effected before bringing the sample into contact with the polymer surface.

2. The process according to claim 1, wherein the polymer is polyalkyl methacrylate, polyvinyl acetate, polycyclohexyl methacrylate or a copolymer comprising units of these polymers.

3. The process according to claim 1 or 2, wherein the surface is a surface of a microtiter plate, cuvette or measuring tube.

4. The process according to any one of claims 1 to 3, wherein the linker is a polyalkylene glycol, polyalkylene imine, polyalkylene amine or polyalkylene sulfide.

5. The process according to any one of claims 1 to 4, wherein the recognition structure is an antigen, a specific antibody, an enzyme or an inhibitor of enzymes.

6. The process according to any one of claims 1 to 5, wherein the sample is a body fluid selected from the group consisting of blood, urine, plasma or sperm.

7. The process according to any one of claims 1 to 6, wherein the polymer is present in the form of particles which are applied to the surface of any material.

8. The process according to claim 1, wherein the surface is the surface of a stirring device which is introduced into the sample receptacle.

9. The process according to claim 8, wherein the labeled substance is color detectable in the visible area.

10. The process according to any one of claims 8 to 9, further comprising the quantification of the components to be detected by means of a color comparison with a comparator scale.

11. The process according to any one of claims 1 to 7, further comprising the quantification of the components to be detected by means of a suitable detection process.
